(19) United States
(12) Patent Application Publication
(10) Pub. No.: US 2025/0345551 A1
(43) Pub. Date: Nov. 13, 2025

(54) SYSTEM AND METHOD FOR REDUCING MOTION SICKNESS

(71) Applicant: HYUNDAI MOBIS CO., LTD., Seoul (KR)

(72) Inventors: Ki Hee PARK, Yongin-si (KR); Yeong Hun PARK, Yongin-si (KR)

(73) Assignee: HYUNDAI MOBIS CO., LTD., Seoul (KR)

(21) Appl. No.: 18/967,983

(22) Filed: Dec. 4, 2024

(30) Foreign Application Priority Data
May 7, 2024 (KR) 10-2024-0060087

(51) Int. Cl. A61M 21/00; G16H 50/30

(52) U.S. Cl. CPC A61M 21/00; G16H 50/30; A61M 2205/3303; A61M 2209/088

(57) ABSTRACT

A method including measuring a biosignal of a passenger in a moving device through a biosensor, acquiring a behavior signal of the moving device from a sensor of the moving device, inputting the measured biosignal and the acquired behavior signal to a processor including a deep learning model, segmenting, by the processor, the input behavior signal into units of segments and labeling the input biosignal, extracting, by the processor, a feature value by fusing the segmented behavior signal and the labeled biosignal, and controlling, by the processor, the moving device by predicting a motion sickness state of the passenger based on the extracted feature value.

This application claims the benefit under 35 USC § 119(a) of Korean Patent Application No. 10-2024-0060087, filed on May 7, 2024, the entire disclosure of which is incorporated herein by reference for all purposes.

### BACKGROUND

### 1. Field

The present disclosure relates to a method and system for reducing motion sickness capable of more effectively controlling a moving device while increasing reliability of prediction of a motion sickness state of a passenger riding in the moving device based on a deep learning model.

### 2. Discussion of the Related Art

Motion sickness may occur when exposed to one or more specific motions for a long period of time. In this instance, factors such as temperature, smell, emotions, and digestion may serve to promote motion sickness.

In particular, many people experience car sickness, and many solutions have been proposed to suppress car sickness. A representative example thereof is a suspension taken before riding in a vehicle, but this suspension contains ingredients such as scopolamine, dimenhydrinate, diphenhydramine, promethazine, and meclizine, and thus have many side effects.

Accordingly, anti-motion sickness patches are mainly used these days. However, since anti-motion sickness patches do not have the same effect on everyone and in all situations, consumers are greatly dissatisfied with effectiveness thereof.

Motion sickness, which is accompanied by dizziness and nausea when riding in a vehicle, is caused by the brain temporarily becoming confused when there is a mismatch in input between sensory organs (visual, somatosensory, semicircular canal, etc.) that maintain balance or detect movement and posture.

A human remembers responses of sensory organs such as eyes and ears to muscle movement in the brain, and prepares for and responds to similar movement thereafter by prediction of the sensory organs using remembered information. However, in a state of riding in a vehicle, there is no muscle movement due to moving, or movement is different from existing memory, so that a mismatch in sensation occurs and motion sickness occurs.

Conventional technology for detecting motion sickness predicted motion sickness of the passenger based on biosignals of the passenger. However, there were limitations in consistently measuring biosignals of the passenger, and thus there was a problem in accurately detecting a motion sickness state of the passenger based on the biosignals of the passenger.

Therefore, a means is needed to accurately predict the motion sickness state of the passenger and control the vehicle based thereon.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In a general aspect, here is provided a method including measuring a biosignal of a passenger in a moving device through a biosensor, acquiring a behavior signal of the moving device from a sensor of the moving device, inputting the measured biosignal and the acquired behavior signal to a processor including a deep learning model, segmenting, by the processor, the input behavior signal into units of segments and labeling the input biosignal, extracting, by the processor, a feature value by fusing the segmented behavior signal and the labeled biosignal, and controlling, by the processor, the moving device by predicting a motion sickness state of the passenger based on the extracted feature value.

The segmenting may include segmenting the input behavior signal using a window size of a preset time unit.

The deep learning model may be constructed according one or more of an RNN (Recurrent Neural Network) to which an LSTM (Long Short-Term Memory) method is applied, a 1D CNN (1-Dimensional Convolutional Neural Network), a 2D CNN (2-Dimensional Convolutional Neural Network), and a CRNN (Convolutional recurrent neural network).

The method may include training the deep learning model based on the extracted feature value.

The method may include controlling, by the processor, one or more of a display, an internal light, an air conditioning device, a seat, a speaker, and a diffuser of the moving device.

The biosensor may include a wearable biosensor configured to be worn by the passenger, the biosensor may measure a biosignal, the biosignal including one or more of EEG, heart rate, electrocardiogram, and pulse of the passenger.

The sensor of the moving device may include one or more of an acceleration sensor, a brake sensor, a tilt sensor, a yaw/pitch/roll sensor, a steering angle sensor, and a GPS sensor.

In a general aspect, here is provided a system including one or more processors configured to execute instructions and a memory storing the instructions, an execution of the instructions configuring the one or more processors to receive a measurement of a biosignal of a passenger of a moving device, the moving device including the one or more processors, acquire a behavior signal of the moving device from a sensor of the moving device, and input the measured biosignal and the acquired behavior signal to a deep learning model, segment the input behavior signal into units of segments, label the input biosignal, extract a feature value by fusing the segmented behavior signal and the labeled biosignal, and control the moving device by predicting a motion sickness state of the passenger based on the extracted feature value.

The biosignal may be obtained from a biosensor and the biosensor may measure a biosignal, the biosignal including one or more of one or more of EEG, heart rate, electrocardiogram, and pulse of the passenger.

The biosensor may include a wearable biosensor configured to be worn by the passenger.

The one or more processors may be configured to segment the input behavior signal using a window size of a preset time unit.

The deep learning model may be constructed according to one or more of an RNN (Recurrent Neural Network) to which an LSTM (Long Short-Term Memory) method is applied, a 1D CNN (1-Dimensional Convolutional Neural Network), a 2D CNN (2-Dimensional Convolutional Neural Network), and a CRNN (Convolutional recurrent neural network).

The processor may be configured to train the deep learning model based on the extracted feature value.

The one or more processors may be configured to control one or more of a display, an internal light, an air conditioning device, a seat, a speaker, and a diffuser of the moving device.

The sensor of the moving device may include one or more of an acceleration sensor, a brake sensor, a tilt sensor, a yaw/pitch/roll sensor, a steering angle sensor, and a GPS sensor.

### BRIEF DESCRIPTION OF THE DRAWINGS

FIG. 1 is a block diagram of a motion sickness reduction system according to an embodiment of the present disclosure;

FIG. 2 is a diagram for more specifically describing an output unit of FIG. 1; and

FIG. 3 is a diagram for describing a method of reducing motion sickness according to an embodiment of the present disclosure.

Throughout the drawings and the detailed description, unless otherwise described or provided, the same, or like, drawing reference numerals may be understood to refer to the same, or like, elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order.

The features described herein may be embodied in different forms and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Advantages and features of the present disclosure and methods of achieving the advantages and features will be clear with reference to embodiments described in detail below together with the accompanying drawings. However, the present disclosure is not limited to the embodiments disclosed herein but will be implemented in various forms. The embodiments of the present disclosure are provided so that the present disclosure is completely disclosed, and a person with ordinary skill in the art can fully understand the scope of the present disclosure. The present disclosure will be defined only by the scope of the appended claims. Meanwhile, the terms used in the present specification are for explaining the embodiments, not for limiting the present disclosure.

Terms, such as first, second, A, B, (a), (b) or the like, may be used herein to describe components. Each of these terminologies is not used to define an essence, order or sequence of a corresponding component but used merely to distinguish the corresponding component from other component(s). For example, a first component may be referred to as a second component, and similarly the second component may also be referred to as the first component.

Throughout the specification, when a component is described as being “connected to,” or “coupled to” another component, it may be directly “connected to,” or “coupled to” the other component, or there may be one or more other components intervening therebetween. In contrast, when an element is described as being “directly connected to,” or “directly coupled to” another element, there can be no other elements intervening therebetween.

In a description of the embodiment, in a case in which any one element is described as being formed on or under another element, such a description includes both a case in which the two elements are formed in direct contact with each other and a case in which the two elements are in indirect contact with each other with one or more other elements interposed between the two elements. In addition, when one element is described as being formed on or under another element, such a description may include a case in which the one element is formed at an upper side or a lower side with respect to another element.

The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

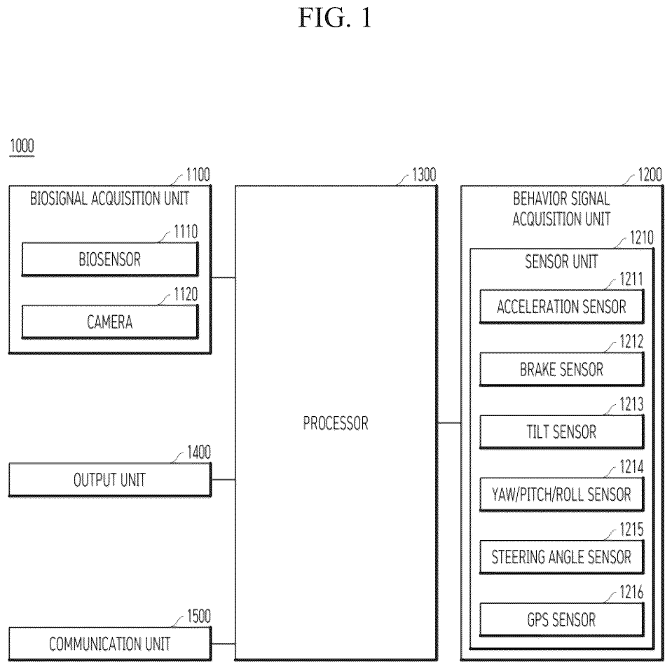

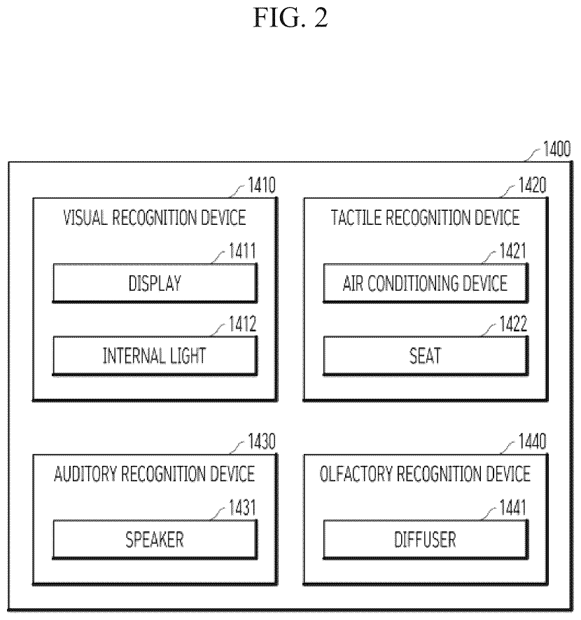

FIG. 1 is a block diagram of a motion sickness reduction system 1000 according to an embodiment of the present disclosure. In addition, FIG. 2 is a diagram for more specifically describing an output unit 1400 of FIG. 1.

The motion sickness reduction system 1000 according to an embodiment of the present disclosure may include a biosignal measurement unit 1100, a behavior signal acquisition unit 1200, a processor 1300, the output unit 1400, and a communication unit 1500.

First, the biosignal measurement unit 1100 may include a biosensor 1110 and a camera 1120. Here, the biosensor 1110 may include a wearable biosensor that may be worn by a passenger. In addition, the biosensor 1110 may include an electroencephalogram (EEG) sensor, an electrocardiogram sensor, a skin conductance sensor, and a respiration detection sensor.

For example, the biosensor 1110 may include an earset for measuring an EEG signal. The earset may be worn around an car of the passenger to measure an EEG signal around a left or right temporal lobe of the passenger. In addition, the biosensor 1110 may include a smartwatch for measuring a photoplethysmogram (PPG) signal. The smartwatch may be worn on a wrist of the passenger to measure a biosignal such as heart rate using a blood flow of the passenger.

Further, the biosensor 1110 described above may be used to acquire a biosignal of at least one of EEG, heart rate, electrocardiogram, or pulse of the passenger. In addition, an image of the passenger may be monitored through the camera 1120 to collect state information such as location of the passenger and temperature change.

That is, the motion sickness reduction system 1000 according to an embodiment of the present disclosure may measure a biosignal including state information of the passenger using the biosignal measurement unit 1100.

The communication unit 1500 may wirelessly transmit a biosignal of the passenger measured using the biosignal measurement unit 1100 to the processor 1300 through communication such as Bluetooth, infrared communication, RFID, or UWB, or may transmit the biosignal of the passenger to the processor 1300 by wire. In addition, the communication unit 1500 may be connected wirelessly or by wire to various devices carried by the passenger.

The behavior signal acquisition unit 1200 may acquire a behavior signal of the moving device from a sensor located in the moving device. Here, a sensor unit 1210 may include an acceleration sensor 1211, a brake sensor 1212, a tilt sensor 1213, a yaw/pitch/roll sensor 1214, a steering angle sensor 1215, and a GPS sensor 1216.

That is, the motion sickness reduction system 1000 according to an embodiment of the present disclosure may acquire a behavior signal of the moving device, such as straight driving, turning, changes in speed, acceleration, and changes in height, through the sensor unit 1210 described above.

As described above, motion sickness, which is accompanied by dizziness and vomiting when riding in the moving device, is caused by a temporary confusion in the brain when there is a mismatch in input between the sensory organs (visual, somatosensory, semicircular canal, etc.) that maintain balance or detect movement and posture. In humans, reactions of the sensory organs such as the eyes and cars to muscle movements are remembered in the brain, and when similar movement occurs later, the sensory organs predict in advance and prepare and react based on the remembered information.

However, in a state of riding in the moving device, there is no muscle movement due to moving, or movement is different from the existing memory, and thus sensory mismatch occurs, causing motion sickness of the passenger of the moving device.

Here, there was a problem that motion sickness of the passenger could not be accurately detected since only vibration information of the moving device, which was extremely limited, was used to detect motion sickness in the past. In addition, conventional technology for detecting motion sickness predicted motion sickness of the passenger based on biosignals of the passenger. However, there were limitations in consistently measuring biosignals of the passenger, and thus there was a problem in accurately detecting a motion sickness state of the passenger based on the biosignals of the passenger.

Accordingly, an object of the motion sickness reduction system 1000 according to an embodiment of the present disclosure is to reduce motion sickness of the passenger by predicting the motion sickness state of the passenger and controlling the moving device using the biosignal measurement unit 1100 that measures a biosignal of the passenger, the behavior signal acquisition unit 1200 that acquires a behavior signal of the moving device, and the processor 1300 including a deep learning model to be described later.

In the motion sickness reduction system 1000 according to an embodiment of the present disclosure, the processor 1300 may include a deep learning model. In addition, a biosignal measured through the biosignal measurement unit 1100 and a behavior signal acquired through the behavior signal acquisition unit 1200 may be input to the deep learning model. In other words, the measured biosignal and the acquired behavior signal may be input values in a motion sickness prediction method based on the deep learning model.

The processor 1300 may segment an input behavior signal into units of sections (epoching), label an input biosignal, and extract a feature value by fusing the segmented behavior signal and the labeled biosignal. In addition, the moving device may be controlled by predicting the motion sickness state of the passenger based on the extracted feature value. Here, the processor 1300 may segment the input behavior signal into units of sections using a window size of a preset time unit.

That is, the motion sickness reduction system 1000 according to an embodiment of the present disclosure may acquire a behavior signal of the moving device through the sensor unit 1210 described above and perform a preprocessing process of scaling and segmentation using a window size. In addition, labeling noise may be minimized by generating a biosignal-based label of the passenger in the behavior signal of the moving device. In addition, the deep learning model may be trained based thereon, and in this way, it is possible to enhance reliability of prediction of the motion sickness state of the passenger based on the deep learning model.

Here, the deep learning model may be constructed based on at least one of an RNN (Recurrent Neural Network) to which an LSTM (Long Short-Term Memory) method is applied, a 1D CNN (1-Dimensional Convolutional Neural Network), a 2D CNN (2-Dimensional Convolutional Neural Network), or a CRNN (Convolutional recurrent neural network).

In the motion sickness reduction system 1000 according to an embodiment of the present disclosure, the output unit 1400 is a device located in the moving device, and may be controlled through the processor 1300. More specifically, referring to FIG. 2, the output unit 1400 may include a visual recognition device 1410, a tactile recognition device 1420, an auditory recognition device (speaker), and an olfactory recognition device 1440.

Here, the visual recognition device 1410 may include a display 1411 and an internal light 1412 including ambient light. The tactile recognition device 1420 may include an air conditioning device 1421 and a seat 1422. The auditory recognition device 1430 may include a speaker 1431, and the olfactory recognition device 1440 may include a diffuser 1441.

Further, the motion sickness reduction system 1000 according to an embodiment of the present disclosure may predict the motion sickness state of the passenger through the processor 1300 so that at least one of the display 1411, the internal light 1412, the air conditioning device 1421, the seat 1422, the speaker 1431, or the diffuser 1441 may be controlled. That is, the motion sickness of the passenger may be reduced through visual, tactile, auditory, and olfactory stimuli recognizable by the passenger.

Meanwhile, the block diagram of the motion sickness reduction system 1000 illustrated in FIGS. 1 and 2 is merely a block diagram for an embodiment of the present disclosure, and each component of the block diagram may be integrated, added, or omitted according to the specifications of the motion sickness reduction system 1000 that is actually implemented. That is, two or more components may be combined into one component, or one component may be divided into two or more components and configured, as needed. In addition, a function performed by each block is for describing an embodiment of the present disclosure, and a specific operation or device thereof does not limit the scope of rights of the present disclosure.

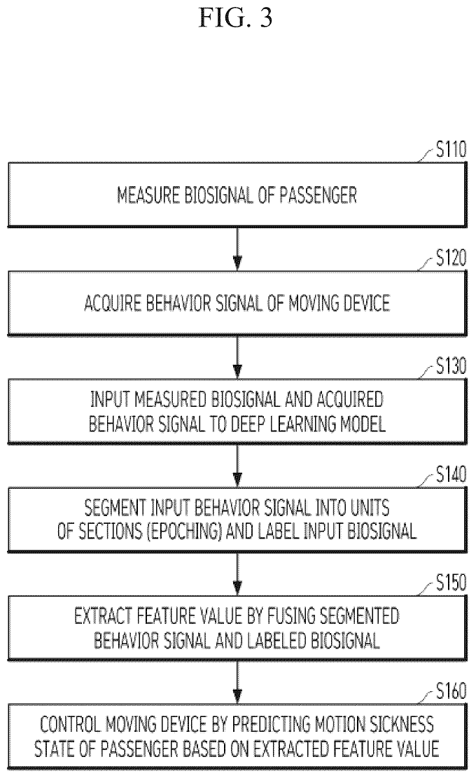

FIG. 3 is a diagram for describing a method of reducing motion sickness according to an embodiment of the present disclosure.

Hereinafter, a description will be given of the method of reducing motion sickness according to the embodiment of the present disclosure by summarizing matters described above with reference to FIGS. 1 and 2.

First, a biosignal of the passenger may be measured through the biosensor 1110 (S110), and a behavior signal of the moving device may be acquired from the sensor of the moving device (S120).

Here, the biosensor 1110 may include a wearable biosensor that may be worn by the passenger. For example, the biosensor 1110 may include an EEG sensor, an electrocardiogram, a skin conductance sensor, a respiration detection sensor, etc.

For example, the biosensor 1110 may include an earset for measuring an EEG signal. The earset may be worn around an car of the passenger to measure an EEG signal around a left or right temporal lobe of the passenger. In addition, the biosensor 1110 may include a smartwatch for measuring a PPG signal. The smartwatch may be worn on a wrist of the passenger to measure a biosignal such as heart rate using a blood flow of the passenger.

Further, the biosensor 1110 described above may be used to acquire a biosignal of at least one of EEG, heart rate, electrocardiogram, or pulse of the passenger. In addition, an image of the passenger may be monitored through the camera 1120 to collect state information such as location of the passenger and temperature change.

In addition, the sensor unit 1410 of the moving device may include an acceleration sensor 1411, a brake sensor 1412, a tilt sensor 1413, a yaw/pitch/roll sensor 1414, a steering angle sensor 1415, and a GPS sensor 1416. In addition, it is possible to acquire a behavior signal of the moving device, such as straight driving, turning, changes in speed, acceleration, and changes in height, through the sensor unit 1410.

In addition, the measured biosignal and the acquired behavior signal may be input to the deep learning model. In other words, the measured biosignal and the acquired behavior signal may be input values in a motion sickness prediction method based on the deep learning model.

Thereafter, the input behavior signal may be segmented into units of sections (epoching), the input biosignal may be labeled (S140), and a feature value may be extracted by fusing the segmented behavior signal and the labeled biosignal (S150). In addition, the motion sickness state of the passenger may be predicted based on the extracted feature value to control the moving device (S160).

In particular, the method of reducing motion sickness according to an embodiment of the present disclosure may acquire a behavior signal of the moving device through the sensor unit 1210 described above and perform a preprocessing process of scaling and segmentation using a window size. In addition, labeling noise may be minimized by generating a biosignal-based label of the passenger in the behavior signal of the moving device. In addition, the deep learning model may be trained based thereon, and in this way, it is possible to enhance reliability of prediction of the motion sickness state of the passenger based on the deep learning model.

To summarize the above description, the method and system for reducing motion sickness according to the present disclosure may more effectively control the moving device while increasing reliability of prediction of the motion sickness state of the passenger riding in the moving device based on the deep learning model. In addition, it is possible to improve motion sickness prediction performance by generating the biosignal-based label of the passenger in the behavior signal of the moving device.

The method and system for reducing motion sickness according to the present disclosure may more effectively control the moving device while increasing reliability of prediction of the motion sickness state of the passenger riding in the moving device based on the deep learning model.

In addition, it is possible to improve motion sickness prediction performance by generating the biosignal-based label of the passenger in the behavior signal of the moving device.

Various embodiments of the present disclosure do not list all available combinations but are for describing a representative aspect of the present disclosure, and descriptions of various embodiments may be applied independently or may be applied through a combination of two or more.

A number of embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method, the method comprising:
measuring a biosignal of a passenger in a moving device through a biosensor;
acquiring a behavior signal of the moving device from a sensor of the moving device;
inputting the measured biosignal and the acquired behavior signal to a processor including a deep learning model;
segmenting, by the processor, the input behavior signal into units of segments and labeling the input biosignal;
extracting, by the processor, a feature value by fusing the segmented behavior signal and the labeled biosignal; and
controlling, by the processor, the moving device by predicting a motion sickness state of the passenger based on the extracted feature value.

2. The method according to claim 1, wherein the segmenting comprises:
segmenting the input behavior signal using a window size of a preset time unit.

3. The method according to claim 1, wherein the deep learning model is constructed according one or more of an RNN (Recurrent Neural Network) to which an LSTM (Long Short-Term Memory) method is applied, a 1D CNN (1-Dimensional Convolutional Neural Network), a 2D CNN (2-Dimensional Convolutional Neural Network), and a CRNN (Convolutional recurrent neural network).

4. The method according to claim 3, further comprising:
training the deep learning model based on the extracted feature value.

5. The method according to claim 1, further comprising:
controlling, by the processor, one or more of a display, an internal light, an air conditioning device, a seat, a speaker, and a diffuser of the moving device.

6. The method according to claim 1, wherein the biosensor comprises a wearable biosensor configured to be worn by the passenger, and
wherein the biosensor measures a biosignal, the biosignal including one or more of EEG, heart rate, electrocardiogram, and pulse of the passenger.

7. The method according to claim 1, wherein the sensor of the moving device comprises one or more of an acceleration sensor, a brake sensor, a tilt sensor, a yaw/pitch/roll sensor, a steering angle sensor, and a GPS sensor.

8. A system, the system comprising:
one or more processors configured to execute instructions; and
a memory storing the instructions, wherein execution of the instructions configures the one or more processors to:
receive a measurement of a biosignal of a passenger of a moving device, the moving device comprising the one or more processors;
acquire a behavior signal of the moving device from a sensor of the moving device;
input the measured biosignal and the acquired behavior signal to a deep learning model;
segment the input behavior signal into units of segments;
label the input biosignal;
extract a feature value by fusing the segmented behavior signal and the labeled biosignal; and
control the moving device by predicting a motion sickness state of the passenger based on the extracted feature value.

9. The system according to claim 8, wherein the biosignal is obtained from a biosensor, and
wherein the biosensor measures a biosignal, the biosignal including one or more of one or more of EEG, heart rate, electrocardiogram, and pulse of the passenger.

10. The system according to claim 9, wherein the biosensor comprises:
a wearable biosensor configured to be worn by the passenger.

11. The system according to claim 8, wherein the one or more processors are further configured to:
segment the input behavior signal using a window size of a preset time unit.

12. The system according to claim 8, wherein the deep learning model is constructed according to one or more of an RNN (Recurrent Neural Network) to which an LSTM (Long Short-Term Memory) method is applied, a 1D CNN (1-Dimensional Convolutional Neural Network), a 2D CNN (2-Dimensional Convolutional Neural Network), and a CRNN (Convolutional recurrent neural network).

13. The system according to claim 12, wherein the processor is further configured to:
train the deep learning model based on the extracted feature value.

14. The system according to claim 8, wherein the one or more processors are further configured to:
control one or more of a display, an internal light, an air conditioning device, a seat, a speaker, and a diffuser of the moving device.

15. The system according to claim 8, wherein the sensor of the moving device comprises one or more of an acceleration sensor, a brake sensor, a tilt sensor, a yaw/pitch/roll sensor, a steering angle sensor, and a GPS sensor.
